(19) United States
(12) Patent Application Publication
(10) Pub. No.: US 2022/0202827 A1
(43) Pub. Date: Jun. 30, 2022

(54) Enhancement of Melanocyte Migration Using ROCK Inhibitors

(71) Applicant: The General Hospital Corporation, Boston, MA (US)

(72) Inventor: David E. Fisher, Newton, MA (US)

(21) Appl. No.: 17/602,076

(22) PCT Filed: Apr. 8, 2020
(86) PCT No.: PCT/US2020/027307

§ 371 Date: Oct. 7, 2021

Related U.S. Application Data
(60) Provisional application No. 62/882,209, filed on Aug. 2, 2019.
(60) Provisional application No. 62/876,073, filed on Jul. 19, 2019.
(60) Provisional application No. 62/830,735, filed on Apr. 8, 2019.

(51) Int. Cl. A61K 31/551; A61K 31/472; A61K 31/4409; A61K 31/7105; A61K 31/519; A61P 17/00

(52) U.S. Cl. CPC A61K 31/551; A61K 31/472; A61P 17/00; A61K 31/7105; A61K 31/519; A61K 31/4409

(57) ABSTRACT

Compositions and methods for stimulating proliferation and/or migration of melanocytes in order to re-pigment skin regions, using ROCK inhibitors and optionally SIK inhibitors.

### CLAIM OF PRIORITY

This application claims the benefit of U.S. Provisional Application Ser. Nos. 62/830,735, filed on Apr. 8, 2019; 62/876,073, filed on Jul. 19, 2019; and 62/882,209, filed on Aug. 2, 2019. The entire contents of the foregoing are incorporated herein by reference.

### TECHNICAL HELD

Provided herein are compositions and methods for stimulating proliferation and/or migration of melanocytes in order to re-pigment these skin regions, using rho associated coiled-coil containing protein kinase (ROCK) inhibitors and optionally SIK inhibitors.

### BACKGROUND

Several skin conditions are notable for absence or deficiencies in melanocyte numbers. One example is vitiligo, a common condition of skin depigmentation that can affect any area of the body.

### SUMMARY

Described herein are methods for stimulating proliferation and/or migration of melanocytes in order to re-pigment skin affected by loss or absence of melanocytes, e.g., vitiligo lesions or other areas of hypopigmentation. The methods can include administration of ROCK inhibitors, which were incidentally discovered to stimulate keratinocytes to produce the melanocyte growth factor SCF, alone or in combination with SIK inhibitors, which were previously described as inducers of melanocyte pigmentation (though not previously tested for proliferation or migration activities) and/or other agents to stimulate melanocyte migration and/or proliferation, thereby treating vitiligo in a subject.

Thus provided herein are methods for treating a subject having a disorder associated with loss or absence of skin pigmentation. The methods include administering to the subject a therapeutically effective amount of an inhibitor rho associated coiled-coil containing protein kinase 1 (ROCK1), ROCK2, or both ROCK1 and ROCK2. Also provided are inhibitors of rho associated coiled-coil containing protein kinase 1 (ROCK1), ROCK2, and/or ROCK1 and ROCK2, for use in a method of treating a subject having a disorder associated with loss or absence of skin pigmentation.

In some embodiments, the subject has vitiligo.

In some embodiments, the inhibitor of ROCK1 is a small molecule inhibitor of ROCK, e.g., fasudil, ripasudil, Netarsudil or Y27632.

In some embodiments, the inhibitor is an inhibitory nucleic acid that targets and specifically reduces expression of ROCK1, or ROCK1 and ROCK2, e.g., a small interfering RNA, small hairpin RNA, or antisense oligonucleotide. In some embodiments, the inhibitory nucleic acid is modified.

In some embodiments, the methods include administering an inhibitor of salt induced kinase (SIK). In some embodiments, the inhibitor of SIK is a small molecule inhibitor of SIK, YKL, 06-061 or YKL 06-062.

In some embodiments, the inhibitor of ROCK (and/or optional inhibitor of SIK) is administered topically to, or by injection into, an area of skin exhibiting a loss or absence of pigmentation.

Also provided herein are compositions comprising an inhibitor of ROCK and an inhibitor of SIK.

In some embodiments, the inhibitor of ROCK1 is a small molecule inhibitor of ROCK. In some embodiments, the small molecule inhibitor of ROCK1 is fasudil, ripasudil, Netarsudil or Y27632.

In some embodiments, the inhibitor of SIK is a small molecule inhibitor of SIK.

In some embodiments, the small molecule inhibitor of SIK is YKL 06-061 or YKL 06-062.

In some embodiments, the composition is formulated for topical application, e.g., as a salve, ointment, gel, lotion, serum, milk, balm, mask, foam, spray, or cream.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-C. Keratinocytes that survive culture in TWA medium with Y-27632 can enhance melanocyte growth through a paracrine signaling pathway. A. Equal numbers of pure passage 3 melanocytes were plated with TWA medium in 4 groups with addition(s) as indicated: 1) Y-27632 (10 μM) alone (Y in graph), 2) passage 3 keratinocytes alone (K), 3) keratinocytes and Y-27632 (10 μM) (K+Y), and 4) no addition (negative control, con). Relative fold changes in melanocyte proliferation compared to the negative control were determined by counting the number of melanocytes at 24 h and 48 h after plating. B. Conditioned TWA media were obtained from the 4 groups of passage 3 melanocyte cultures in (A) at 48 h after plating. Then, equal numbers of passage 3 melanocytes were plated in 4 groups, each with one of the conditioned medium. Relative fold changes in melanocyte proliferation compared to the negative control were determined by counting the number of melanocytes at 24 h and 48 h after plating. C. RT-PCR analysis of potential melanocyte growth-enhancing factors that have been reported to be secreted from cultured human keratinocytes, at 0, 12, 24, and 48 h after culture of passage 3 keratinocytes with TWA medium.

FIGS. 2A-E. Y-27632 can increase SCF expression in keratinocytes, which can promote melanocyte growth. A. RT-PCR analysis of SCF mRNA expression in passage 3 keratinocytes grown in K-SFM with (Y) or without (con) Y-27632 for the indicated times. **p<0.01 comparing Y-treated with the corresponding control group. B. Western blot analysis of SCF protein expression with or without Y-27632 for 24, 48 or 72 h. C. RT-PCR analysis of SCF mRNA expression in keratinocytes grown in K-SFM with increasing concentrations of Y-27632 for 48 h. **p<0.01, ***p<0.005 comparing Y-27632-treated with untreated cells (0). D. gRT-PCR analysis of SCF mRNA expression in keratinocytes grown in K-SFM, 72 h after transfection of ROCK1 and ROCK2 siRNAs individually and together. **p<0.01 when compared with the control cells transfected with scramble siRNA (siCtrl). E. Passage 3 keratinocytes were cultured in K-SFM with or without 1′-27632 for 48 h, and then the conditioned media were collected. The conditioned medium with Y-27632 was treated with an SCF antibody or control rabbit IgG, or was untreated, while the conditioned medium without Y-27632 remained untreated (control). The media were then cultured with passage 3 melanocytes for 48 h. Relative fold changes in melanocyte proliferation compared to the control medium were determined by counting the numbers of melanocytes. *p<0.05, **p<0.01 compared with the control; #p<0.05 comparing the SCF-treated group with the IgG-treated group.

FIGS. 3A-C Both Rock and SIK inhibitors could enhances melanocyte migration and combination of both inhibitors produces a synergistic effect. A. Representative images of migrated melanocytes (dark grey) in the transwell migration assay with different conditions as indicated. B. Quantification of number of migrated melanocytes (The number of cells that had migrated into was counted in 5 randomly selected high-power microscopic fields), statistical analysis (student t test) showed **p<0.01 when compared to the control group. C. The transwell migration assay was performed under the indicated conditions. ROCKi: Rock inhibitor Y-27632 (10 μM), SIKi: SIK inhibitors with different concentrations as indicated.

### DETAILED DESCRIPTION

Several skin conditions are notable for absence or deficiencies in melanocyte numbers. One example is vitiligo, a common condition of skin depigmentation. This disclosure describes strategies aimed at stimulating proliferation and/or migration of melanocytes in order to re-pigment these skin regions. The present methods include the use of ROCK inhibitors, which were incidentally discovered as described herein to stimulate keratinocytes to produce the melanocyte growth factor SCF (Ilachiya et al. J Invest Derm. April 2001, 116(4):578-586), with or without SIK inhibitors, which were previously described as inducers of melanocyte pigmentation (Mujahid et al., Cell Rep. 2017 Jun. 13; 19(11): 2177-2184).

Methods of Treatment

Provided herein are methods for the treatment of disorders associated with loss or absence of skin pigmentation caused by a loss or absence of functional melanocytes (melanin-producing cells) in the skin. In some embodiments, the disorder is vitiligo. Other disorders include hypopigmentation, e.g., caused by chemical exposure or formation of scar tissue after an injury. Generally, the methods include administering a therapeutically effective amount of a ROCK inhibitor (e.g., a small molecule or inhibitory nucleic acid, e.g., as described herein) to a subject who is in need of, or who has been determined to be in need of, such treatment. In some embodiments, the methods include administering (e.g., concurrently or consecutively) a therapeutically effective amount of a SIK inhibitor (e.g., a small molecule or inhibitory nucleic acid, e.g., as described herein). The ROCK inhibitor and SIK can be administered together (e.g., at substantially the same time, in the same or different compositions), or can be administered at different times, e.g., one before the other, on the same or different schedules.

As used in this context, to “treat” means to ameliorate at least one symptom of the disorder associated with loss or absence of skin pigmentation. For example, a treatment can result in a reduction in size, growth, or appearance of an area of loss or absence of skin pigmentation (lesion), and a return or approach to normal pigmentation. Administration of a therapeutically effective amount of a compound described herein for the treatment of a condition associated with loss or absence of skin pigmentation will result in decreased number of lesions, frequency of appearance of lesions, or reduced likelihood of recurrence in the same or other locations. The methods can also include application to a lesion, or to an area of skin where a lesion was previously present (e.g., to reduce the risk of recurrence), or to an area of skin where a lesion has not yet appeared (e.g., the face, to reduce the risk of appearance of a lesion).

The application can be topical or by injection into the lesion.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as is the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Small Molecule Inhibitors of ROCK

A number of small molecule inhibitors of ROCK1/2 are known in the art, many of which are commercially available. For example, the following small molecule inhibitors of ROCK1, ROCK2, or ROCK1 and 2 can be used: cyclohexanecarboxamides such as Y-27632 ((+)-(R)-trans-4-(1-aminoethyl)-N-(4-pyridyl)cyclohexanecarboxamide dihydrochloride) and Y-30131 ((+)-(R)-trans-4-(1-aminoethyl)-N-(1H-pyrrolo[2,3-b]pyridin-4-yl)cyclohexanecarboxamide dihydrochloride) (see Ishizaki et al., Mol Pharmacol. 2000 May; 57(5):976-83); dihydropyrimidinones and dihydropyrimidines, e.g., bicyclic dihydropyrimidine-carboxamides (such as those described in Sehon et al. J. Med. Chem., 2008, 51 (21): 6631-6634 and US2018/0170939); ureidobenzamides such as CAY10622 (3-[[[[[4-(aminocarbonyl) phenyl]amino]carbonyl]amino]methyl]-N-(1,2,3,4-etrahydro-7-isoquinolinyl)-benzamide); Thiazovivin; GSK429286A; RKI-1447 (1-(3-Hydroxybenzyl)-3-(4-(pyridin-4-yl)thiazol-2-yl)urea); GSK180736A (GSKI 80736); Hydroxyfasudil. (HA-1100); OXA 06; Y-39983; Netarsudil (AR-13324, see Lin et al., J Ocul Pharmacol Ther. 2018 Mar. 1; 34(1-2): 40-51, U.S. Pat. Nos. 8,450,344 and 8,394,826); GSK269962/GSK269962A; Fasudil (HA-1077, 1-(5-isoquinolinesulfonyl)-homopiperazine) and its derivatives such Ripasudil. (K-115, 4-fluoro-5-[[(2S)-2-methyl-1,4-diazepan-1-yl]sulfonyl]isoquinoline; see WO1999/20620) and others that share the core structure of 5-0,4-diazepan-1-ylsulfonypisoquinoline; KD025 (SLx-2119) and related compound and XD-4000 (see, e.g. Liao et aL 2007 Cardiovasc Pharamcol 50:17-24; WO2010/104851 US 2012/0202793); SR 3677; AS 1892802; 1-1-1152 ((S)-(+)-2-Methyl-1-[(4-methyl-5-isoquinolinyl)sulfonyl]homopiperazine, ikenoya et al., J. Neurochem. 81:9, 2002; Sasaki et al., Pharmacol. Ther. 93:225, 2002); N-(4-Pyridyl)-N′-(2,4,6-trichlorophenyOurea (Takami et al., Bioorg. Med. Chem. 12:2115, 2004); and 3-(4-Pyridyl)-1H-indole (Yarrow et al., Chem. Biol. 12:385, 2005); 3-[2-(aminomethyl)-5-[(pyridin-4-yl)carbamoyl]phenyl] benzoates including AMA0076 (compound 32, Boland et al., Bioorganic & Medicinal Chemistry Letters 23(23): 6442-6446 (2013)) TC-S 7001 and A1713148, and pharmaceutically acceptable salts thereof. Inhibitors with the scaffold 4-Phenyl-1H-pyrrolo [2,3-b] pyridine,including compound TS-122, are described in Shen et al., Scientific Reports 5:16749 (2015). Other ROCK inhibitors include isoquinoline sulfonyl derivatives disclosed in WO 97/23222, Nature 389, 990-994 (1997) and WO 99/64011; heterocyclic amino derivatives disclosed in WO 01/56988; indazole derivatives disclosed in WO 02/100833; pyridylthiazole urea and other ROCK1 Inhibitors as described in 20170049760; and quinazoline derivatives disclosed in WO 02/076976 and WO 02/076977; in WO02053143, p. 7, lines 1-5, EP1163910 A1, p. 3-6, WO02076976 A2, p. 4-9, preferably the compounds described on p. 10-13 and p. 14 lines 1-3, WO02/076977A2, the compounds I-VI of p. 4-5, WO03/082808, p. 3-p. 10 (until line 14), the indazole derivates described in U.S. Pat. No. 7,563,906 B2, WO2005074643A2, p. 4-5 and the specific compounds of p. 10-11, WO2008015001, pages 4-6, EP1256574, claims 1-3, EP1270570, claims 1-4, and EP 1 550 660. These inhibitors are generally commercially available, e.g., from Santa Cruz Biotechnology, Selleck Chemicals, and Tocris, among others. For example, fasudil and Hydroxy fasudil are obtainable from Asahi Kasei Pharma Corp (PMID: 3598899), Y-39983 is obtainable from Novartis/Senju (PMID: 11606042) and Y27632 is obtainable from Mitsubishi Pharma (PrvHD: 9862451). (S)-(+)-2-Methyl-1-[(4-methyl-5-isoquinolinyl) sulfonyl]hornopiperazinel, N-(4-Pyridyl)-N¹-(2,4,6-trichlorophenyl) urea and 3-(4-Pyridyl)-1H-indole are also available at AXXORA (UK) Ltd and other suppliers.

Additional small molecule Rho kinase inhibitors include those described in PCI Publication Nos. WO2013030216; WO2007042321A2; WO2008049919; WO2011023986A1; WO2011107608A1; WO2003059913, WO2003064397, WO2005003101, WO22004112719, WO 2009/155209; WO 2012/135697; WO 2005/003101; WO2003062225; WO 98/06433; and WO2003062227; US. Pat. Nos. 7,217,722; 7,199,147; 8,071,779; 8,093,266; 7,199,147; 6,369,087; 6,369,086; 6,372,733; 8,637,310; 9,174,939; 6,372,778B1; European Patents and applications 2628482, 1256578; 1270570; 1550660; EP0370498A2; and EP0721331A1; and U.S. Patent Application Publication Nos. 2016/0237095; 2015/0238601; 2014/0336440; 2014/0179689; 2013/0131106; 2012/0178752; 2011/0166104; 2010/0183604; 2010/0041645; 2008/0161297; 2012/0270868; 2009/0203678; 2010/0137324; 2013/0131059; 2003/0220357, 2006/0241127, 2005/0182040 and 2005/0197328. See also Tamura et al. Biophys Ada 2005 1754:245-252; Defect and Boland, Expert Opin Ther Pat 27 507-515 (2017); Pan et al., Drug Discovery Today 18(23-24):1323-1333 (2013); Lin and Zheng, Expert Opinion on Drug Discovery, 10(9):991-1010 (2015); US20180110837.

Small Molecule Inhibitors of SIK

A number of small molecule inhibitors of SIK are known in the art, many of which are commercially available. For example, the following small molecule inhibitors of SIK can be used: a 2,4-diaminopyrimidine compound as described in U.S. Pat. No. 9,670,165; macrocyclic compounds of Formula (I), bicyclic urea compounds of Formula (II), (III), and (IV), and compounds of Formula (V), (VI), (VI-A), or (VII) SIK inhibitors disclosed in WO2018/160774; or SIK inhibitors described in WO2018053373. Exemplary SIK inhibitors include HG-01-11-02, HG-10-15-03, HG-10-150-02, HG-10-32-01, HG-10-62-01, HG-10-88-02, HG-10-93-01, HG-11-123-01, HG-11-136-01, HG-11-137-01, HG-11-139-01, HG-11-139-02, HG-11-143-01, HG-11-6-02, HG-9-120-01, HG-9-148-01, HG-9-150-02, HG-9-87-02, HG-9-91-01, YKL-04-103, YKL-04-104, YKL-04-105, YKL-04-106, YKL-04-107, YKL-04-108, YKL-04-112, YKL-04-113, YKL-04-114, YKL-04-115, YKL-04-118, YKL-04-125, YKL-04-136-1, YKL-04-136-10, YKL-04-136-11, YKL-04-136-2YKL-04-136-3, YKL-04-136-4, YKL-04-136-5, YKL-04-193-01, YKL-04-193-02, YKL-05-120, YKL-05-200-1, YKL-05-200-2, YKL-05-201-1, YKL-05-201-2, YKL-05-203-1, YKL-05-203-2, YKL-05-204-1, YKL-05-204-2, YKL-06-029, YKL-06-058, YKL-06-059, YKL-06-060, YKL-06-061, YKL-06-062YKL-06-29, YKL-06-30, YKL-06-31 YKL-06-33, YKL-06-46, YKL-06-50. In some embodiments, the SIK inhibitor is HG-9-91-01, HG-11-137-01, HG-11-139-02, YKL-05-099, YKL-05-200-2, YKL-05-201-1, YKL-05-204-1, YKL-06-029, YKL-06-059, YKL-06-060, YKL-06-06 YKL-06-062, ARN-3236, Pterosin B, or MRT199665. In some embodiments, the SIK inhibitor is YKL-05-120, YKL-05-200-1, YKL-05-200-2, YKL-05-201-1, YKL-05-201 -2, YKL-05-203-1, YKL-05-203-2, YKL-05-204-1, YKL-05-204-2, YKL-06-029, YKL-06-058, YKL-06-059, YKL-06-060, YKL-06-061, YKL-06-062, YKL-06-29, YKL-06-30, YKL-06-31, YKL-06-33, YKL-06-46, YKL-06-50, HG-11-136-01, HG-11-137-01, 11G-11-139-01, HG-11-139-02, HG-9-91-01, or YKL-04-108. In preferred embodiments, the SIK inhibitor is YKL 06-061 or YKL 06-062. See, e.g., Mujahid et al., Cell Rep. 2017 Jun. 13; 19(11): 2177-2184; WO2018160774; and WO2018053373.

Inhibitory Nucleic Acids

Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics that specifically hybridize to at least a portion of a target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof.

Exemplary mRNA target sequences for ROCK1 (rho associated coiled-coil containing protein kinase 1) are provided in GenBank at Acca. No. NM_005406.2. Exemplary mRNA target sequences for SIK (salt inducible kinase 1) are provided in GenBank at Acc. No. NM_173354.5.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the target sequence. For example, a specific functional region can be targeted, e.g., a region comprising a known functional region (e.g., a promoter region). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a target sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this invention, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA, (ssDNA). In a most preferred embodiment, hybridization will occur at 42° C. in 250 mM NaCl, 25 mM trisodium citrate, 1% SDS, 50% formamide and 200 mg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within a target RNA. For example, an antisense compound in which 18 of 20 nucleobases of the antisense oligonucleotide are complementary, and would therefore specifically hybridize, to a target region would represent 90 percent complementarity Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656), Inhibitory nucleic acids that hybridize to an RNA can be identified through routine experimentation. In general, the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

Modified Inhibitory Nucleic Acids

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleotides and/or oligonucleotide mimetics as described above.

Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, ˜N(CH₃)˜CHO˜₂ (known as a methylene(tnethylimino) or MMI backbone], CH₂—O—N(CH₃)—CH₂, CH₂—N(CH₃)—N (CH₃)—CH₂ and O—N(CH₃)—CH₂—CH₂ backbones, wherein the native phosphodiester backbone is represented as O—P—O—CH,; amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2¹; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid oligonucleotide mimetics are described in Wang of al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholine linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596, 086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH; OCH₃, OCH₃ O(CH₂)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3 OCF₃; O-, S-, or N-alkyl; O-, S-, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-0-CH₂CH₂OCH₃, also known as 2′-O-(2-methoxyethyl)] (Martin et al, Helv. Chim. Acta, 1995, 78, 486). Other preferred modifications include 2′-methoxy (2′-0-CH₃), 2′-propoxy (2′OCH₂CH₂CH₃) and 2′-fluoro (2′F). Similar modifications may also be made at other positions on the oligonucleotide, particularly the 3′ position of the sugar on the 3′ terminal nucleotide and the 5′ position of 5′ terminal nucleotide. Oligonucleotides may also have sugar miinetics such as cyclobutyls in place of the pentoffiranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C). 5-hydroxytnethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine; 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T, and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and auanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino; 8-thiol, 8-thioalkyl, 8-hydroxyl and other 8-substituted adenines and guanines, 5-halo particularly 5- bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science and Engineering’, pages 858-859, Kroschwitz, J. I., ed, John Wiley & Sons, 1990, those disclosed by Englisch et al., Angewandle Chemie, International Edition', 1991, 30, page 613, and those disclosed by Sanghvi, Y. S., Chapter 15, Antisense Research and Applications', pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purifies, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. Nos. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity,cellular distribution, or cellular uptake of the oligonucleotide. Such moieties comprise but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4. 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18; 3777-3783), a polyamine or a polyethylene glycol chain (Mancharan et al., Nucleosides 8: Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthioi, a thiocholesterol, an aliphatic chain, e.g., dodecandiol or undecyl residues, a phospholipid, e.g., di-hexadecyl-rac-glycerol or triethylammonium1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105, 5; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Locked Nucleic Acids (LNAs)

In some embodiments, the modified inhibitory nucleic acids used in the methods described herein comprise locked nucleic acid (LNA) molecules, e.g., including [alpha]-L-LNAs. LNAs comprise ribonucleic acid analogues wherein the ribose ring is “locked” by a methylene bridge between the 2′-oxgygen and the 4′-carbon i.e., oligonucleotides containing at least one LNA monomer, that is, one 2′-O,4t-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herien.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com), See, e,g., You et al., Nuc. Acids. Res. 34:e60 (2006); McTigue et al., Biochemistry 43:5388-405 (2004); and Levin et al., Nuc. Acids. Res. 34:e14 (2006). For example, “gene walk” methods, similar to those used to design antisense oligos, can be used to optimize the inhibitory activity of the LNA; for example, a series of oligonucleotides of 10-30 nucleotides spanning the length of a target RNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) oligonucleotides). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

Making and Using Inhibitory Nucleic Acids

The nucleic acid sequences used to practice the methods described herein, whether RNA, cDNA, genomic DNA,vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g. in vitro, bacterial, fungal, mammalian, yeast, insect or plant cell expression systems.

Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants).

Nucleic acid sequences used to practice this invention can be synthesized in vitro by well-known chemical synthesis techniques, as described in, e.g., Adams (1983) J. Am. Chem. Soc. 105:661; Belousov (1997) Nucleic. Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethyiaminoethyl (2′-O-DMADE), 2′-O-dimethylaminopropyl (2′-O-DMAP), 2′-O-dimethylaminoethyloxyethyl (2′-O-DMAEOE), or 2′-O-N-methylaceta.mido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2(3):287-290 (2005); Koshkin et al., J. Am. Chem, Soc., 120(50)13252-13253 (1998)). For additional modifications see US 20100004320, US 20090298916, and US 20090143326.

Techniques for the manipulation of nucleic acids used to practice this invention, such as, e.g., subcloning, labeling probes (e.g., random-primer labeling using Mellow polymerase, nick translation, amplification), sequencing, hybridization and the like are well described in the scientific and patent literature, see, e.g., Sambrook et al., Molecular Cloning; A Laboratoty Manual 3d ed. (2001); Current Protocols in Molecular Biology, Ausubel et al., eds. (John Wiley & Sons, Inc., New York 2010); Kriegler, Gene Transfer and Erpression: A Laboratory Manual (1990); Laboratory Techniques In Biochemistry And Molecular Biology: Hybridization With Nucleic Acid Probes, Part 1. Theory and Nucleic Acid Preparation, Tijssen, ed. Elsevier, N.Y. (1993).

Pharmaceutical Compositions and Methods of Administration

The methods described herein include the use of pharmaceutical compositions comprising an inhibitor of ROCK as an active ingredient, e.g., small molecules or inhibitory nucleic acid sequences designed to target a ROCK RNA. In some embodiments, supplemental active compounds can be included, e.g., SIK inhibitors (e.g., small molecules or inhibitory nucleic acid sequences designed to target a SrK RNA as described herein).

Pharmaceutical compositions typically include a pharmaceutically acceptable carrier. As used herein the language “pharmaceutically acceptable carrier” includes to saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal or topical, transmucosal, and rectal administration. s

Methods of formulating suitable pharmaceutical compositions are known in the art, see, e.g., Remington: The Science and Practice of Pharmacy, 21st ed., 2005; and the books in the series Drugs and the Pharmaceutical Sciences: A Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite, chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal or topical administration, the active compounds are formulated into ointments, salves, gels, lotions, foams, serums, milks, balms, masks, sprays, or creams as generally known in the art.

The pharmaceutical compositions can also be prepared in the form of suppositories (e.g., with conventional suppository bases such as cocoa butter and other glycerides) or retention enemas for rectal delivery.

In some embodiments, compositions for topical application can further comprise cosmetically-acceptable carriers or vehicles and any optional components. A number of such cosmetically acceptable carriers, vehicles and optional components are known in the art and include carriers and vehicles suitable for application to skin (e.g., sunscreens, foams, ointments, salves, gels, balms, creams, milks, lotions, masks, serums, sprays, etc.), see, e.g., U.S. Pat. Nos. 6,645,512 and 6,641,824. In particular, optional components that may be desirable include, but are not limited to absorbents, anti-acne actives, anti-caking agents, anti-foaming agents, anti-fungal actives, anti-inflammatory actives, anti-microbial actives, anti-oxidants, antiperspirant/deodorant actives, anti-skin atrophy actives, anti-viral agents, anti-wrinkle actives, artificial tanning agents and accelerators, astringents, barrier repair agents, binders, buffering agents, bulking agents, chelating agents, colorants, dyes, enzymes, essential oils, film formers, flavors, fragrances, humectants, hydrocolloids, light diffusers, nail enamels, opacifying agents, optical brighteners, optical modifiers, particulates, perfumes, pH adjusters, sequestering agents, skin conditioners/moisturizers, skin feel modifiers, skin protectants, skin senates, skin treating agents, skin exfoliating agents, skin lightening agents, skin soothing and/or healing agents, skin thickeners, sunscreen actives, topical anesthetics, vitamin compounds, and combinations thereof.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

Materials and Methods

The following materials and methods were used in the Examples below.

Isolation and Culture of Primary Melanocytes and Keratinocytes

Foreskin tissues were trimmed to remove the fat layer and then incubated with Dispase solution (2.5 mg/ml in PBS, overnight at 4° C.). On the second day, the epidermis was peeled from the dermis with fine forceps and chopped into small pieces, incubated with 0.05% trypsin for 15-30 min, neutralized with 10% FBS in DMEM, filtered (100 mm filter, Millipore), centrifuged, and rinsed with PBS. In the conventional method of melanocyte isolation, the epidermal cells were plated into culture vessels with melanocyte culture medium TIVA (Ham's F12, Mediatech, Inc., Herndon, Va., USA; 10% fetal bovine serum; 1× penicillin/streptomycin/glutamine, Invitrogen, Carlsbad, Calif., USA; 1×10⁻⁴ M 3-isobutyl-1-methyl xanthine (IBMX), Sigma, St Louis, Mo., USA; 50 ng/ml 12-O-tetradecanoyl phorbol-13-acetate (TPA), Sigma; 2 μM Na₃VO₄; 1×10⁻³ M N⁶,2′-O-dibutyryladenosine 3′,5′-cyclic monophosphate (dbcAMP), Sigma) (Halaban et al., 2000; Yokoyama et al., 2008). The culture medium was changed once every 2 days until confluency was reached. For the new method, the isolated melanocytes were seeded with TIVA medium plus 10 μM Y-27632 (Y0503, Sigma) for two days, then the culture medium was replaced with standard TIVA without Y-27632 every 2 days, as in the conventional method. For quantification of melanocyte number during the initial culture (passage 0), the cultured cells were collected after incubation with 0.05% trypsin at 37° C. for only 3 minutes, which did not detach the keratinocytes, and counted using a TC20™ automated cell counter (Bio-Rad). For isolation of keratinocytes, the dissociated epidermal cells were seeded with keratinocyte culture medium (K-SFM, Gibco/Thermo Fisher Scientific, Waltham, Mass., USA) into culture dishes pretreated with coating matrix containing type-I collagen (Gibco, R-011-K) and the medium was changed every 2 days. In order to understand the role of Y-27632 in promoting melanocyte growth, different media were used in different experiments depending on the experimental needs; information about the medium and protocol used for each experiment is listed in the following table.

| Cells plated | Plating medium and subsequent protocol |
|---|---|
| Dissociated | Plated with TIVA ± Y-27632 and incubated for 48 h, followed by removal of Y-27632 from the |
| epidermis | treated groups and continuation of incubation |
| Dissociated | Plated with TIVA + Y-27632 and incubated for a number of days before removal of Y-27632. |
| epidermis | Incubation continued until Day 12 for all groups. |
| Passage 3 | Plated with TIVA ± Y-27632 and incubated |
| melanocytes | |
| Passage 3 | Plated with TIVA ± Y-27632 and incubated for 48 h |
| melanocytes | |
| Dissociated | Plated with TIVA ± Y-27632 and incubated for 48 h |
| epidermis | |
| Passage 3 | Plated with TIVA ± Y-27632 and incubated for 24 h |
| keratinocytes | |
| keratinocyte- | Plated with TIVA ± Y-27632 and incubated for 48 h |
| melanocyte | |
| aggregates | |
| Passage 3 | Plated with TIVA in dishes precoated with keratinocytes or uncoated and incubated for 1 or 7 |
| melanocytes | days |
| Passage 3 | Plated with TIVA ± Y-27632 and incubated for 24 h |
| keratinocytes | |
| Passage 3 | Plated with K-SFM and incubated 24 h, switched to TIVA ± Y-27632 and incubated for another |
| keratinocytes | 24 h |
| Passage 3 | Plated with TIVA ± Y-27632 and incubated for 24 h, followed by removal of Y-27632 from the |
| keratinocytes | treated group and continuation of incubation for another 24 h or 48 h (total of 48 h or 72 h) |
| Dissociated | Plated with TIVA ± Y-27632 and incubated for 48 h, followed by removal of Y-27632 from the |
| epidermis | treated group and continuation of incubation for another 3 or 12 days (total of 5 or 14 days) |
| Dissociated | Plated in collagen-coated dishes with either TIVA ± Y-27632 or K-SFM ± Y-27632 and incubated |
| epidermis | for two days, followed by replacement of medium for every group with TIVA alone and |
| | continuation of incubation for up to a total of 15 days after plating. |
| Passage 3 | Plated with TIVA ± Y-27632 and ± passage 3 keratinocytes (4 groups) and incubated for 24 h or |
| melanocytes | 48 h. |
| Passage 3 | Plated with conditioned TIVA media and incubated for 24 h or 48 h |
| melanocytes | |
| Passage 3 | Plated with TIVA and incubated for the indicated times |
| keratinocytes | |
| Dissociated | Plated with TIVA and incubated for 96 h, with Y-27632 present for the first 48 h of incubation, |
| epidermis | the second 48 h, all 96 h, or not at all. |
| Passage 3 | Plated with K-SFM ± Y-27632 and incubated for 24-72 h |
| keratinocytes | |
| Passage 3 | Plated with K-SFM ± Y-27632 and incubated for 24-48 h |
| keratinocytes | |
| Passage 3 | Plated with K-SFM and grown to 60% confluency, then transfected with siRNAs and incubated |
| keratinocytes | an additional 72 h |
| Passage 3 | Keratinocytes were plated with K-SFM ± Y-27632 and incubated for 48 h, then the conditioned |
| keratinocytes, | media were treated. Melanocytes were then plated with the conditioned media and incubated |
| Passage 3 | for 48 h. |
| melanocytes | |
| Passage 3 | Plated with TIVA ± SCF and incubated for 12-60 h |
| melanocytes | |
| Dissociated | Plated with TIVA + Y-27632 and incubated for 48 h, followed by removal of Y-27632 and |
| epidermis, | subsequently incubating and passaging the cells with TIVA alone (new method). |
| Passage 1 | |
| melanocytes | |
| Passage 2 | Conventional method: Plated and incubated with TIVA alone |
| melanocytes | |
| Passage 3 | Isolated by conventional or new method as above: 24 h after plating at passage 3, treated with |
| melanocytes | forskolin or DMSO vehicle for 48 h |
| Passage 3 | Melanocytes isolated by conventional or new method as above. |
| melanocytes & | Keratinocytes isolated by conventional method and cultured in SFM, Dermal fibroblasts were |
| keratinocytes | cultured in DMEM with 10% FBS. |
| Passage 3 | Plated with TIVA ± Y-27632 and incubated for 60 min |
| melanocytes | |
| Dissociated | Plated with TIVA ± Y-27632, incubated for two days, then switched to TIVA alone for 3 more |
| epidermis | days |
| Dissociated | Left panel: Plated with TIVA + Y-27632, incubated for two days, then switched to TIVA alone |
| epidermis | until passaged with K-SFM (P1 keratinocytes)Right Panel: Plated and passaged with K-SFM |
| Dissociated | Plated with TIVA ± Y-27632 for indicated times |
| epidermis | |
| Passage 3 | Plated with K-SFM and grown to 60% confluency, then transfected with siRNAs and incubated |
| keratinocytes | an additional 72 h |
| Dissociated | Plated with TIVA containing Y-27632 or SCF or PBS (control). After two days: replacement of |
| epidermis | TIVA + Y-27632 with TIVA alone; continuation of SCF in TIVA + SCF group throughout. |

qRT-PCR Analysis

Total RNA was extracted from cells using a QIAGEN RNeasy Plus Mini Kit (QIAGEN, Hilden, Germany) according to manufacturer's instructions. The RNAs were dissolved in nuclease-free water and the concentrations were measured with a Nanodrop spectrophotometer. qRT-PCR was carried out in 12.5 μl reaction volumes using a KAPA SYBR FAST One-Step Universal Kit (KAPA Biosystems, Wilmington, Mass., USA) with an ABI 7500 Fast System programmed as follows: 42° C. for 5 min, 95° C. for 1 min, and 40 cycles of PCR at 95° C. for 15 s and 60° C. for 30 s. Data were acquired and analyzed with 7500 Fast System SDS software (Life Tedmologies, Grand Island, N.Y., USA). The primers for each assessed gene are listed below; 36B4 was used as a housekeeping gene for the internal control.

ET-1:
F:
(SEQ ID NO: 1)
5′-CAGCAGTCTTAGGCGCTGAG-3′,
R:
(SEQ ID NO: 2)
5′-ACTCTTTATCCATCAGGGACGAG-3′;
FGF2:
F:
(SEQ ID NO: 3)
5:-ATGGCAGCCGGGAGCATCACCCACG-3′,
R:
(SEQ ID NO: 4)
5′-TCAGCTCTTCGCAGACATTGGAAG-3′;
POMC:
F:
(SEQ ID NO: 5)
5′-GAGGGCAAGCGCTCCTACTCC-3′,
R:
(SEQ ID NO: 6)
5′-GGGGCCCTCGTCCTTCTTCTC-3′;
NOF:
F:
(SEQ ID NO: 7)
5′-CACACTGAGGTGCATAGCGT-3′,
R:
(SEQ ID NO: 8)
5′-TGATGACCGCTTGCTCCTGT-3′;
GM-CST:
F:
(SEQ ID NO: 9)
5′-CTGGAGAACGAAAAGAACGAAGAC-3′,
R:
(SEQ ID NO: 10)
5′-TCAAAAGGGATATCAAACAGAAAG-3′;
SCF (KITLG):
F:
(SEQ ID NO: 11)
5′-AAGAGGATAATGAGATAAGTATGTTGC-3′,
R:
(SEQ ID NO: 12)
5′-TTACCAGCCAATGTACGAAAGT-3′;
36B4:
F:
(SEQ ID NO: 13)
5′-GCAATGTTGCCAGTGTCTGT-3′,
R:
(SEQ ID NO: 14)
5′-GCCTTGACCTTTTCAGCAAG-3′.

Cell Profferation Assay

Proliferation of melanocytes and keratinocytes was analyzed using Cell Counting Kit-8 (CCK-8, Dojindo Molecular Technologies, Rockville, Md.) according to the manufacturer's specifications. Briefly, cells were seeded at a density of 10⁴ per well in 96-well plates, incubated for 24 h, and then stimulated for 12-60 h with Y-27632 (10 μM) or sterile water vehicle. After treatment, 10 μl of CCK-8 solution was added to each well, the plates were incubated at 37° C. for 1 h, and then the OD value at 450 nm of each well was read on a microplate reader (Multiskan, Thermo Fisher Scientific, USA) to determine the cell viability. The assay was repeated three times.

### Example 1

### Rock Inhibitor (Y-27632) Promote Passaged Melanocyte Growth Through Effect on Keratinocyte

To test whether Y-27632 can enhance the yield of primary melanocytes, dissociated cells isolated from epidermis were plated with melanocyte culture medium (TWA) containing 10 μM Y-27632 and incubated for 48 h, and then the medium was replaced with TIVA without Y-27632. We observed more melanocytes in cultures treated with Y-27632 than in cultures without Y-27632. The differences were significant at 5 or more days after plating, and at 16 days, about 5 times more melanocytes were recovered from Y-27632-treated cultures compared with untreated cultures. We further tested whether longer treatment with Y-27632 had the same effect. We found that continuous treatment with Y-27632 for up to 12 days increased the yield of melanocytes relative to the untreated group, with 4 days of treatment generating the highest yield.

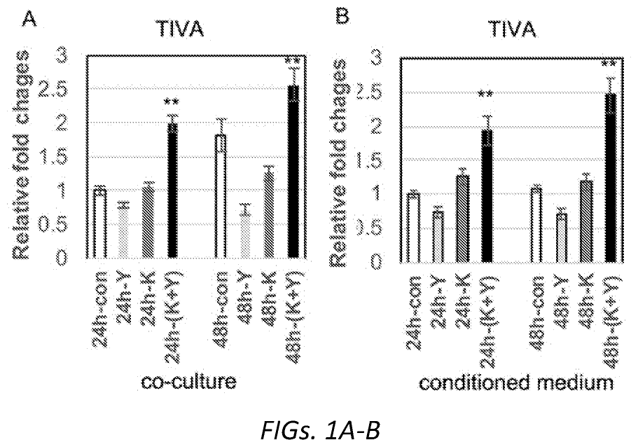

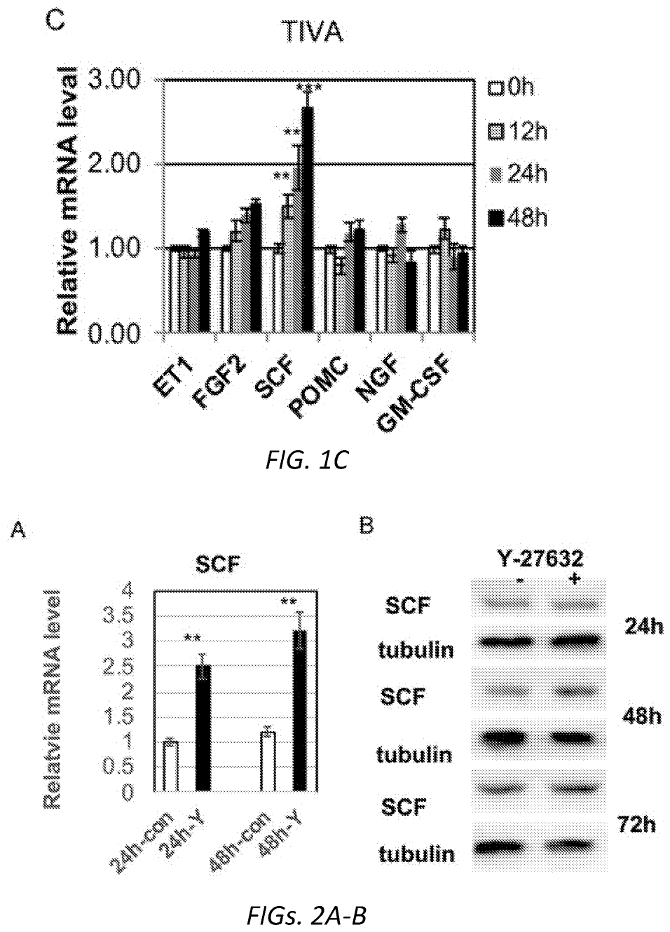

Next, we characterized the enhancement of melanocyte growth by keratinocytes in a co-culture assay. We found that, in TWA medium, neither Y-27632 alone nor keratinocytes medium alone could promote the growth of melanocytes, as the keratinocytes did not survive in TIVA medium without Y-27632. However, the presence of both keratinocytes and Y-27632 (K+Y) could significantly increase melanocyte proliferation (FIG. 1A). Previous studies have clearly demonstrated that cultured keratinocytes can secret arowth factors that enhance the proliferation of melanocytes. Therefore, we hypothesized that the surviving keratinocytes in TIVA medium with Y-27632 can promote proliferation of melanocytes by secreting growth factors into the medium. This hypothesis was confirmed when conditioned medium collected from cultures of keratinocytes in TIVA+Y-27632 significantly enhanced melanocyte proliferation, while medium collected from cultures of melanocytes with TIVA and either keratinocytes or Y-27632 alone, as expected, did not increase melanocyte proliferation (FIG. 1B). These data suggest that Y-27632 can promote melanocyte growth by stimulating keratinocytes to produce one or more growth factors. To identify potential growth factors, quantitative RT-PCR analysis was performed for expression of six factors that have been reported to be secreted by cultured human keratinocytes and could enhance melanocyte growth. Of those six factors, only SCF (KITLG) expression increased significantly in the presence of TIVA+Y-27632 (FIG. 1C).

### Example 2

### Y-27632 Increases SCF Expression in Keratinocytes, Which Enhances the Growth of Melanocytes

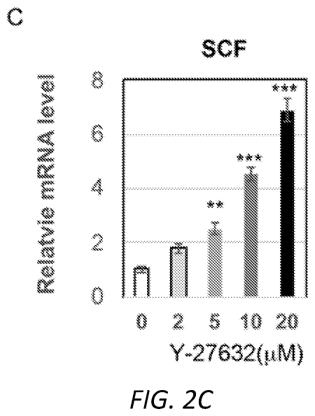

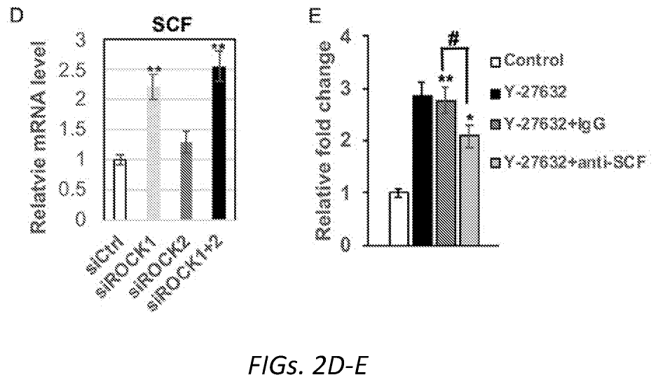

To test whether Y-27632 can enhance expression of SCF in keratinocytes, we cultured keratinocytes in the keratinocyte culture condition (K-SFM) with or without Y-27632 and measured SCF expression. We found that Y-27632 can increase both mRNA and protein levels of SCF in keratinocytes (FIGS. 2A, B), and in a dose-dependent manner (FIG. 2C). To test whether the increased expression of SCF was due to on-target inhibition of ROCK function by Y-27632, we targeted the mRNAs for ROCK isoforms ROCK1 and ROCK2 with previously validated siRNAs (Chang et al., 2018); We found that SCF expression was induced by knockdown of ROCK1 alone or ROCK1 and ROCK2 together, but not by knockdown of ROCK2 alone, suggesting that Y-27632 induces SCF expression mainly through inhibition of ROCK1 (FIG. 2D).

To further confirm that the secretion of SCF protein plays a crucial role in the enhancement of melanocyte growth by keratinocytes in the presence of Y-27632, we cultured keratinocytes in K-SFM with or without Y-27632 for 48 h and then collected the conditioned media for feeding melanocyte cultures. Portions of the Y-27632-treated medium were treated with an SCF antibody or control IgG before culturing with melanocytes. As shown in FIG. 2E, the enhancement of melanocyte growth by Y-27632 was partially inhibited by pretreatment of the conditioned medium with anti-SCF. These data suggest that Y-27632-induced production of SCF by keratinocytes plays an important role in the enhancement of melanocyte proliferation, but keratinocyte-derived factors other than SCF or the keratinocytes themselves also likely contribute additionally.

### Example 3

### Y-27632 Promotes Melanocyte Migration, Which is Further Enhanced by Combining with SIK Inhibitor

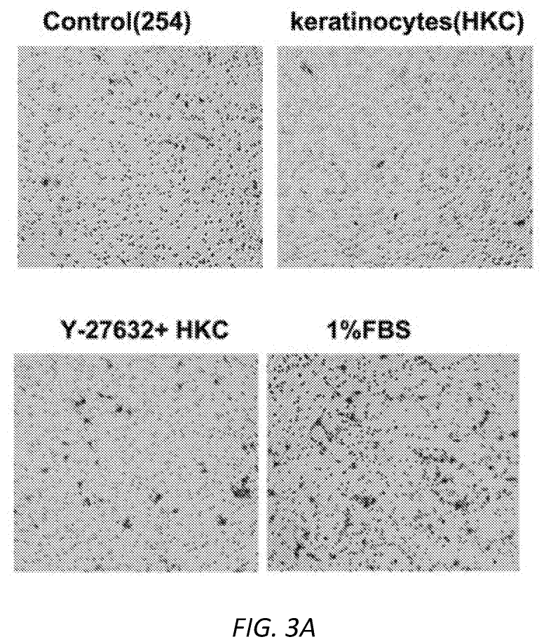

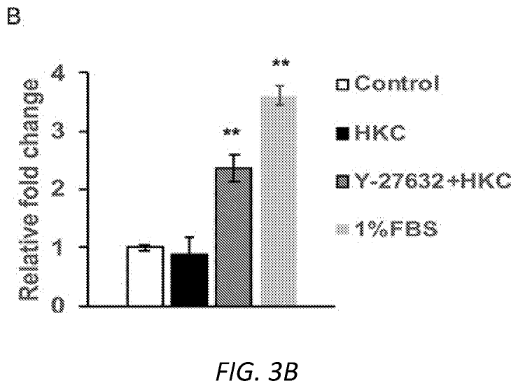

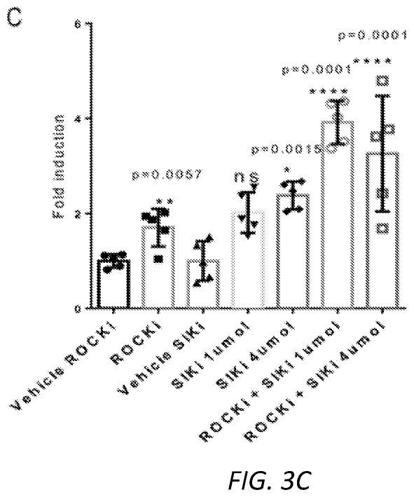

To test the effect of Y-27632 on melanocyte cell migration, 1×10⁵ cells were cultured in the upper chamber of a transwell migration apparatus. The lower chamber was supplemented either with 254 (melanocyte culture medium) without FBS (control) or 254 media plus seeding human keratinocytes (HKC) in the bottom or 254 medium with lOmM Y-27632 plus seeding keratinocytes in the bottom (Y-27632+HKC) or 254 medium in the presence of 1% FBS. The chambers were incubated for 24 hours at 37° C. After removal of non-migrated cells on top of the filter, cells that had migrated through the membrane were fixed in 4% paraformaldehyde washed and then stained with 0.1% crystal violet (FIG. 3A). The number of cells that had migrated through the membrane was counted in six randomly selected high-power microscopic fields and the average number of cells were shown in FIG. 3B. FIG. 3B showed that Y-27632 combined with HKCs significantly induced the migration of melanocytes. We further did a similar migration assay with the different conditions indicated in FIG. 3C to test whether a SIK inhibitor (SIKi), which regulates melanocyte differentiation, or the combination of STK and ROCK inhibitor (ROCKi, 10 uM Y-27632), could enhance melanocyte migration. The results, in FIG. 3C, showed that the combination of SIKi and ROCKi produced the biggest effect on promoting melanocyte migration.

### OTHER EMBODEVIENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject having a disorder associated with loss or absence of skin pigmentation, the method comprising administering to the subject a therapeutically effective amount of an inhibitor rho associated coiled-coil containing protein kinase 1 (ROCK1).

2. The method of claim 1, wherein the subject has vitiligo.

3. The method of claim 1, wherein the inhibitor of ROCK1 is a small molecule inhibitor of ROCK.

4. The method of claim 3, wherein the small molecule inhibitor of ROCK1 is fasudil, ripasudil, Netarsudil or Y27632.

5. The method of claims 1, wherein the inhibitor is an inhibitory nucleic acid that targets and specifically reduces expression of ROCK1, or ROCK1 and ROCK2.

6. The method of claim 5, wherein the inhibitory nucleic acid is a small interfering RNA, small hairpin RNA, or antisense oligonucleotide.

7. The method of claim 5, wherein the inhibitory nucleic acid is modified.

8. The method of claim 1, wherein the inhibitor of ROCK is administered topically to, or by injection into, an area of skin exhibiting a loss or absence of pigmentation.

9. The method of claim 1, further comprising administering an inhibitor of salt induced kinase (SIK).

10. The method of claim 9, wherein the inhibitor of SIK is a small molecule inhibitor of SIK.

11. The method of claim 10, wherein the small molecule inhibitor of SIK is YKL 06-061 or YKL 06-062.

12.-21. (canceled)

22. A composition comprising an inhibitor of ROCK and an inhibitor of SIK.

23. The composition of claim 22, wherein the inhibitor of ROCK1 is a small molecule inhibitor of ROCK.

24. The composition of claim 23, wherein the small molecule inhibitor of ROCK1 is fasudil, ripasudil, Netarsudil or Y27632.

25. The composition of claim 22, wherein the inhibitor of SIK is a small molecule inhibitor of SIK.

26. The composition of claim 25, wherein the small molecule inhibitor of SIK is YKL 06-061 or YKL 06-062.

27. The composition of claim 22, which is formulated for topical application.

28. The composition of claim 22, which is a salve, ointment, gel, lotion, serum, milk, balm, mask, foam, spray, or cream.


SEQUENCE LISTING (14 sequences)

SEQ ID NO: 1 - DNA, 20 bp, Artificial Sequence
  ET-1 Forward Primer
cagcagtcttaggcgctgag

SEQ ID NO: 2 - DNA, 23 bp, Artificial Sequence
  ET-1 Reverse Primer
actctttatccatcagggacgag

SEQ ID NO: 3 - DNA, 25 bp, Artificial Sequence
  FGF2 Forward Primer
atggcagccgggagcatcacccacg

SEQ ID NO: 4 - DNA, 24 bp, Artificial Sequence
  FGF2 Reverse Primer
tcagctcttcgcagacattggaag

SEQ ID NO: 5 - DNA, 21 bp, Artificial Sequence
  POMC Forward Primer
gagggcaagcgctcctactcc

SEQ ID NO: 6 - DNA, 21 bp, Artificial Sequence
  POMC Reverse Primer
ggggccctcgtccttcttctc

SEQ ID NO: 7 - DNA, 20 bp, Artificial Sequence
  NGF Forward Primer
cacactgaggtgcatagcgt

SEQ ID NO: 8 - DNA, 20 bp, Artificial Sequence
  NGF Reverse Primer
tgatgaccgcttgctcctgt

SEQ ID NO: 9 - DNA, 24 bp, Artificial Sequence
  GM-CSF Forward Primer
ctggagaacgaaaagaacgaagac

SEQ ID NO: 10 - DNA, 24 bp, Artificial Sequence
  GM-CSF Reverse Primer
tcaaaagggatatcaaacagaaag

SEQ ID NO: 11 - DNA, 27 bp, Artificial Sequence
  SCF (KITLG) Forward Primer
aagaggataatgagataagtatgttgc

SEQ ID NO: 12 - DNA, 22 bp, Artificial Sequence
  SCF (KITLG) Reverse Primer
ttaccagccaatgtacgaaagt

SEQ ID NO: 13 - DNA, 20 bp, Artificial Sequence
  36B4 Forward Primer
gcaatgttgccagtgtctgt

SEQ ID NO: 14 - DNA, 20 bp, Artificial Sequence
  36B4 Reverse Primer
gccttgaccttttcagcaag